(19) United States
(12) Patent Application Publication
(10) Pub. No.: US 2003/0143658 A1
(43) Pub. Date: Jul. 31, 2003

(54) Rapid methods and devices for the detection of coliform and the detection and confirmation of E. coil

(76) Inventors: Linda J. Richardson Casella, Neuilly sur-Seine (FR); Hermanus Clemens Johannes Gerardus van Balen, Heidevenstraat (NL)

Correspondence Address: Brian D. Voyce, 8401 Sterling Bridge Road, Chapel Hill, NC, 27516, US

(21) Appl. No.: 10/058,393

(22) Filed: Jan. 28, 2002

(51) Int. Cl. C12Q 1/04

(52) U.S. Cl. 435/34

(57) ABSTRACT

The present invention relates to methods and devices for the detection of coliform and for the detection and confirmation of E. coli. In particular, the methods comprises contacting a sample so as to allow any coliform present in the sample to access a growth encouraging medium, incubating the sample at a temperature of at least 37 degrees C. so as to support growth of any coliform that may be present and for a time sufficient to allow growth that can be detected by a fluorogen or chromagen present in the medium, and inspecting the sample for a signal. Also disclosed are a novel antibiotic-free medium and devices containing this medium, both useful in the present methods.

### TECHNICAL FIELD

The present invention relates to methods and devices for the detection of coliform and for the detection and confirmation of E. coli. In particular, the methods comprises contacting a sample so as to allow any coliform present in the sample to access a growth encouraging medium, incubating the sample at a temperature of at least 37 degrees C. so as to support growth of any coliform that may be present and for a time sufficient to allow growth that can be detected by a fluorogen or chromagen present in the medium, and inspecting the sample for a signal. Also disclosed are a novel antibiotic-free medium and devices containing this medium, both useful in the present methods.

### BACKGROUND ART

The detection of coliforms and, in particular, the detection and confirmation of E. coli is of vital public health interest in the areas of potable water testing (including bottled water or beverages) and food safety testing. The art has used enzymatically-driven chromagens or fluorogens to aid in this testing.

One example is the potable or environmental water test disclosed in U.S. Pat. No. 6,063,590 to Brenner el alia. A target sample is placed in a broth containing three components, namely, an ingredient that encourages and repairs injured coliforms, a gram positive cocci suppressing agent, and a non-coliform gram negative anti-bacterial. In a preferred embodiment, both a fluorogen and a chromagen are used. A sample is incubated at 35 degrees C.

Other tests are based on the use of certain enzymatically sensitive substrates (2-nitrophenyl-β-D-galactopyranoside and 4-methylumbelliferyl-β-D-glucuronide) to test for certain coliform related enzymes (β-galactosidase and β-glucuronidase). U.S. Pat. No. 4,923,804 to Ley et alia discloses the use of β-glucuronides for E. coli testing.

### DISCLOSURE OF THE INVENTION

The present invention is related to a method for detecting coliform and for detecting and confirming E. coli coliform in a sample. The method comprises four general steps. First, one contacts the sample with a coliform growth medium in an amount effective to support coliform growth so as to allow any coliform present in the sample to access the medium. Along with conventional growth coliform components, three other selective growth components make up the medium, namely, at least one pH buffer so as to maintain a pH of at least 6.0, at least one coliform sensitive chromagen, and at least one coliform sensitive fluorogen. Next, one incubates the sample at a temperature above 37 degrees C. for a time sufficient to allow coliform growth preferentially over non-coliform growth. Finally, one inspects the sample for a fluorescent or color signal. Preferentially, the sample is incubated at a temperature of at least about 42 degrees C. Typically, one would not incubate above about 44 degrees C.

The present method can also be used for detecting either coliform or E.coli. In the former case, one uses a medium as set forth above

An object of the present invention is to provide a rapid (less than 24 hour, preferably less than 12 hour) test method for the detection of coliform and for the detection and confirmation of generic E. Coli, particularly in food samples.

Another object of the invention is to provide a confirmation test for E. coli without the need for additional testing.

Another object of the invention is to eliminate the requirement to include a selective gram-positive bacteria antibiotic.

For the purposes of the present invention, a “chromagen” includes any substance that either changes color or is colorless and produces a color when acted upon by a biologically related component (such as an enzyme). Also, a “fluorogen” includes any substance that exhibits fluorescence when acted upon by a biologically related component (such as an enzyme).

### PREFERRED MODES OF PRACTICING THE INVENTION

### Selective Growth Medium

Medium useful for the present invention comprises two different enzyme substrates, one for coliforms (4-methylumbelliferyl-β-D-galactopyranoside at 0.1 g/l) and one for E. coli (indoxyl-β-D-glucuronide at 320 μg/ml) in a selective base agar that favors their growth. The selective base agar can be selected from known growth ingredients. A preferred embodiment uses bacterial growth promoters (such as proteose peptone #3 (5.0 g/l) and, yeast extract (3.0 g/l)), an inducer (such as β-D-lactose or lactose) (1.0 g/l)), buffering salts (such as sodium chloride (7.5 g/l), potassium hydrophosphate (3.3 g/l), and sodium dihydrophosphate (1.0 g/l)), gram positive inhibiting salts (such as sodium laurylsulfate (0.2 g/l) and sodium desoxycholate (0.1 g/l)), and agar (15 g/l).

Use of an inducer in the above medium is optional.

Use of antibiotic in the above medium is optional. This novel antibiotic-free medium is substantially less costly than prior art medium including the antibiotic.

Use of agar in the above medium is also optional. This medium can be used either in a most probable number method or absorbent pads

### Comparative Testing on Inoculated Samples

A series of tests were conducted to test and compare the present method on samples contaminated by inoculation with pure strains of E. coli with the prior art Brenner et alia method. A high bio-burden protein, fat, and, sugar rich medium was prepared from fresh meats that had been contaminated or challenged with naturally occurring pseudomonas, lactobacillus, and spore forming bacillus species as a control. In addition, some of the sample was inoculated with one of two pure E coli strains, namely ATCC 25922 or ATCC 35218.

The E. coli strains were incubated for eighteen hours at 37 degrees C. in 5 ml of Tryptone Soya Broth (Merck KgaA, Darmstadt, Germany). The broth was then diluted to an appropriate dilution of 10³ CFU/ml. The final concentration of E. coli suspension was estimated with a dilution ranged poured plate method and plate count agar and/or standard methods agar according to conventional methods approved for the food industry.

The high bio-burden medium was inoculated with an a pure E. coli strain by diluting tenfold twenty grams of test sample with saline peptone solution. A ten ml aliquot of a 10³ CFU/ml dilution of the E. coli strain is added to the sample suspension and stomached for five minutes. One ml of the diluted suspension fluid was inoculated into a 9 cm Petri dish. Fifteen ml of the above-described sterile growth medium ((with and without an antibiotic, namely Cefsulodin) was also introduced.

After eighteen hours of incubation, the samples were analyzed for development of a dark blue color from the chromagen and a fluorescent halo from the fluorogen. Colonies with only the halo are counted as coliform, with those having that halo and the color are counted as E. coli. The percent recovery rate was determined by identification and counting of specific colonies on each plate medium divided by the CFU's found on the standard (Plate Count Agar from Merck KgaA). Preferably, one can view color and fluorescence development using a UV long light (366 to 400 nm) or a normal black light lamp.

The following tables show the results of the comparative testing:

TABLE 1
ATCC 25922 Inoculated Samples

| | Incubation | Recovery | |
|---|---|---|---|
| Medium | T (° C.) | rate (%) | Remarks |
| Cefsulodin | 37 | 61 | Faint color, hard to distinguish |
| | | | fluorescence, visible growth |
| | | | of other microorganisms |
| No Cefsulodin | 37 | 62 | Faint color, hard to distinguish |
| | | | fluorescence, visible growth |
| | | | of other microorganisms |
| Cefsulodin | 42 | 89 | Clear color and fluorescence, |
| | | | no visible growth of |
| | | | other microorganisms |
| No Cefsulodin | 42 | 89 | Clear color and fluorescence, |
| | | | no visible growth of |
| | | | other microorganisms |

(Natural contamination levels before inoculation of pure strain specific E. coli spiking were a total count of 3.10⁸ CFU/g and an E. coli count at less than 10 CFU/g.)

TABLE 2
ATCC 35218 Inoculated samples

| | Incubation | Recovery | |
|---|---|---|---|
| Medium | T (° C.) | rate (%) | Remarks |
| Cefsulodin | 37 | 62 | Faint color, hard to distinguish |
| | | | fluorescence, visible growth |
| | | | of other microorganisms |
| No Cefsulodin | 37 | 64 | Faint color, hard to distinguish |
| | | | fluorescence, visible growth |
| | | | of other microorganisms |
| Cefsulodin | 42 | 79 | Clear color and fluorescence, |
| | | | no visible growth |
| | | | of other microorganisms |
| No Cefsulodin | 42 | 77 | Clear color and fluorescence, |
| | | | no visible growth |
| | | | of other microorganisms |

(Natural contamination levels before inoculation of pure strain specific E. coli spiking were a total count of 6.10⁸ CFU/g and an E. coli count at less than 40 CFU/g.)

With either E. coli strain, the recovery rate and the visual detection is better using the present incubation temperature, i.e., elevated above the industrial standard of 37 degrees C. Moreover, detection is not impaired if the antibiotic is removed from the medium, representing a significant cost savings.

The ordinarily skilled artisan can appreciate that the present invention can incorporate any number of the preferred features described above.

All publications or unpublished patent applications mentioned herein are hereby incorporated by reference thereto.

Other embodiments of the present invention are not presented here which are obvious to those of ordinary skill in the art, now or during the term of any patent issuing from this patent specification, and thus, are within the spirit and scope of the present invention.

We claim:

1. A method for detecting coliform and for detecting and confirming E. coli coliform in a sample comprising:
a) contacting the sample with a medium comprising a growth encouraging medium in an amount effective to support coliform growth, at least one pH buffer so as to maintain a pH of at least 6.0, at least one coliform sensitive chromagen, and at least one coliform sensitive fluorogen, so as to allow any coliform present in the sample to access the medium;
b) incubating the sample at a temperature above 37 degrees C. for a time sufficient to allow coliform growth preferentially over non-coliform growth; and
c) inspecting the sample for a signal.

2. The method of claim 1 wherein the sample is incubated at a temperature of at least about 42 degrees C.

3. A method for detecting coliform in a sample comprising:
a) contacting the sample with a medium comprising a growth encouraging medium in an amount effective to support coliform growth, at least one pH buffer so as to maintain a pH of 6.5 to 8, and at least one coliform sensitive fluorogen, so as to allow any coliform present in the sample to access the medium;
b) incubating the sample at a temperature above 37 degrees C. for a time sufficient to allow coliform growth preferentially over non-coliform growth; and
c) inspecting the sample for a signal.

4. The method of claim 3 wherein the sample is incubated at a temperature of at least 42 degrees C.

5. A method for detecting E. coli coliform in a sample comprising:
a) contacting the sample with a medium comprising a growth encouraging medium in an amount effective to support E. coli coliform growth, at least one pH buffer so as to maintain a pH of at least 6.0, at least one E. coli coliform sensitive chromagen, so as to allow any E. coli coliform present in the sample to access the medium;
b) incubating the sample at a temperature at above 37 degrees C. for a time sufficient to allow coliform growth preferentially over non-coliform growth; and
c) inspecting the sample for a signal.

6. The method of claim 5 wherein the sample is incubated at a temperature of at least 42 degrees C.

7. A medium for detecting coliform and for detecting and confirming E. coli coliform comprising:
a) an antibiotic-free growth encouraging medium in an amount effective to support coliform growth;
b) at least one pH buffer so as to maintain a pH of at least 6.0;
c) at least one coliform sensitive chromagen; and
d) at least one coliform sensitive fluorogen.

8. The medium of claim 7 also comprising an agent for increasing viscosity.

9. The medium of claim 8 wherein the viscosity agent is agar.

10. The medium of claim 7 wherein the elements are in a powdered form.

11. A device for detecting coliform and for detecting and confirming E. coli coliform comprising an absorbent material and the medium of claim 7 adsorbed or placed onto the membrane.

12. The device of claim 11 wherein the medium also comprises an agent for increasing viscosity.

13. The device of claim 12 wherein the viscosity agent is agar.

14. The device of claim 11 wherein the medium elements are in a powdered form.

15. A device for detecting coliform and for detecting and confirming E. coli coliform comprising the medium of claim 7 placed into a growth plate having a plurality of separate chambers.

16. The device of claim 15 wherein the medium also comprises an agent for increasing viscosity.

17. The device of claim 16 wherein the viscosity agent is agar.

18. The device of claim 15 wherein the medium elements are in a powdered form.
